(19) United States
(12) Patent Application Publication
(10) Pub. No.: US 2023/0023566 A1
(43) Pub. Date: Jan. 26, 2023

(54) SYSTEM AND METHOD FOR PROVIDING A NOTIFICATION OF A MEDICAL CONDITION

(71) Applicant: Drägerwerk AG & Co. KGaA, Lübeck (DE)

(72) Inventors: Ryan FORDE, Pelham, NH (US); Devin WEIDNER, Litchfield, NH (US)

(73) Assignee: Drägerwerk AG & Co. KGaA, Lübeck (DK)

(21) Appl. No.: 17/790,327

(22) PCT Filed: Dec. 29, 2020
(86) PCT No.: PCT/IB2020/062521

§ 371 Date: Jun. 30, 2022

Related U.S. Application Data
(60) Provisional application No. 62/955,597, filed on Dec. 31, 2019.

(51) Int. Cl. A61B 5/0205; A61B 5/00; G16H 10/60

(52) U.S. Cl. CPC A61B 5/0205; A61B 5/746; A61B 5/4842; A61B 5/742; G16H 10/60

(57) ABSTRACT

A system and method for preliminarily identifying a medical condition in a monitored patient. A predetermined set of patient physiological parameters that are indicative of the presence of a medical condition is monitored. If all of the patient physiological parameters meet a predetermined criteria, a notification is activated that is indicative of the presence of the medical condition. Optionally, users are provided with guidance concerning additional patient physiological parameters to be checked to confirm the presence of the medical condition.

### BACKGROUND

Current patient monitoring devices commonly include, for example, a heart rate monitor, an electrocardiogram (“EKG”) monitor, and an oxygen saturation (“SpO2”) monitor. Current patient monitoring systems integrate measurement of many physiological parameters of patients (e.g. EKG, SpO2, etc.) into a single patient monitoring device. Such a device includes the patient connections necessary to measure the physiological parameters measurable by the device and a display device which can display the measured physiological parameters in an appropriate manner. Such patient monitors may be considered to be partitioned into two sections. A first operational section controls the reception of signals from the electrodes or probes connected to the patient and performs the signal processing necessary to calculate the desired physiological parameters. A second control, status, and communication section interacts with a user to receive control information and with the first operational section receives the physiological parameters and displays status information and values of the physiological parameters in an appropriate manner. Either or both of these sections may include a computer or processor to control the operation of that section.

Patient monitors are often connected to a central hospital computer system via a hospital network. In this manner, data representing patient physiological parameters may be transferred to the central hospital computer system for temporary or permanent storage in a storage device. Data received from the patient monitors may also be monitored by a person, such as a nurse or other healthcare worker, at the central location. The stored data may be retrieved and analyzed by other healthcare workers via the hospital network. Patient monitors in such a networked system include a terminal which is capable of being connected to and communicating with the hospital network. In such a patient monitor, the control, status and communication section controls the display of the physiological parameters, and also the connection to the hospital network and the exchange of the physiological parameters with other systems, such as other patient monitors, patient treatment devices (e.g., therapeutic devices), patient diagnostic devices, and/or the central computer storage device, via the hospital network.

A common functionality of these systems is to provide a notification when a patient physiological parameter is outside of a predetermined range. For example, a notification could be activated if a patient's heart rate fall below a minimum set point or above a maximum set point. Examples of types of notifications include a flashing indicator light, alternating background colors on a graphical user interface, and an audible alarm. Such notifications are commonly used in combination. Often the predetermined range can be set by a user via the graphical user interface.

The primary purpose of such notifications is to alert hospital staff that the patient requires attention. Such notifications are typically tied to patient physiological parameters that could be indicative of a number of different medial conditions. For example, low SpO2 levels could be a result of cardia arrest, asthma, or sleep apnea.

Some hospital systems are capable of displaying a patient “score”, which is designed to provide an indication of the patient's condition which may require closer attention by hospital staff if the score drops below predetermined minimum. Such patient scores are typically calculated based on a combination of continuously measured patient physiological parameters, but do not provide an indication or notification of a particular medical condition.

There are some medical conditions that can be effectively diagnosed when a combination of patient physiological parameters each meet a criteria. Other medical conditions can only be effectively diagnosed by identifying a plurality of patient physiological parameters, as well as other factors that are not currently able to be monitored or measured using patient monitoring systems. For example, the family medical history, response to administration of a medication, or the results of a laboratory test. For some of these conditions, early diagnosis of the medical condition can improve patient outcomes. Therefore, there is a need for hospital systems to provide hospital staff with an early warning of possible onset of certain medical conditions that are not identifiable from a single patient physiological parameter or patient “score.”

### BRIEF DESCRIPTION OF THE DRAWINGS

For a more complete understanding of the present invention, reference is made to the following detailed description of embodiments considered in conjunction with the accompanying drawings, in which:

FIG. 1 is a block diagram illustrating an exemplary hospital system;

FIG. 2 is a more detailed block diagram illustrating the system illustrated in FIG. 1;

FIG. 3 is a flow chart illustrating a method of providing a automated notification of the possible existence of a medical condition if multiple patient physiological parameters are outside of preset limits;

FIG. 4 is a flow chart illustrating a specific implementation of the method of FIG. 3 to provide a preliminary automated diagnosis for malignant hyperthermia;

FIG. 5A shows an exemplary graphical user interface in an operating room for the patient monitoring system of FIG. 1;

FIG. 5B shows the graphical user interface of FIG. 5A in which a notification for malignant hyperthermia has been activated; and

FIG. 5C shows the graphical user interface of FIG. 5A in which a notification for malignant hyperthermia has been activated and parameters that are used to determine the preliminary automated diagnosis have been visually emphasized.

### DETAILED DESCRIPTION

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign particular meaning. Further, it should be noted that, as recited in the specification and in the claims appended hereto, the singular forms “a,” “an,” and “the” include the plural referents unless otherwise stated. Additionally, the terms “comprises” and “comprising” when used herein specify that certain features are present in that embodiment, but should not be interpreted to preclude the presence or addition of additional features, components, operations, and/or groups thereof.

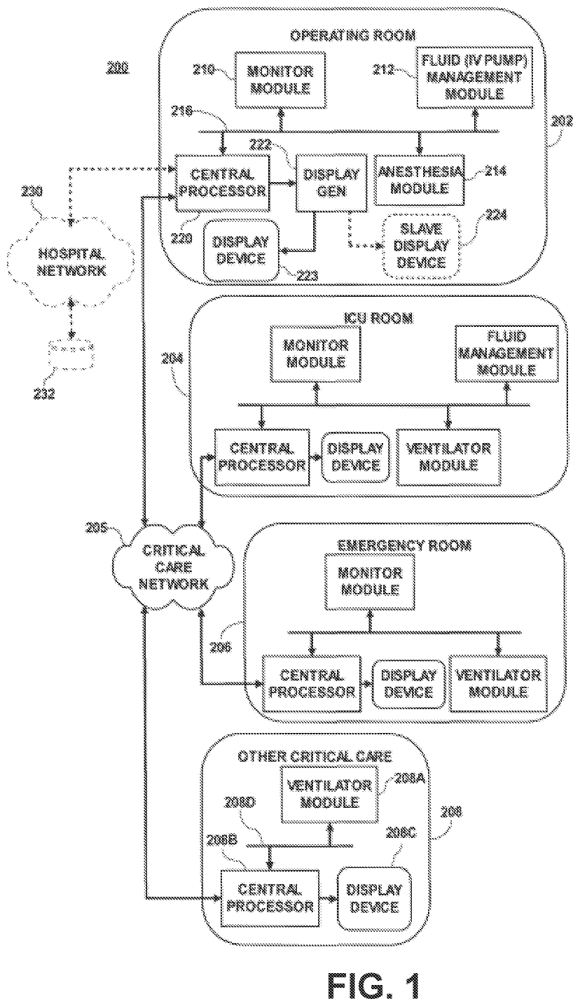

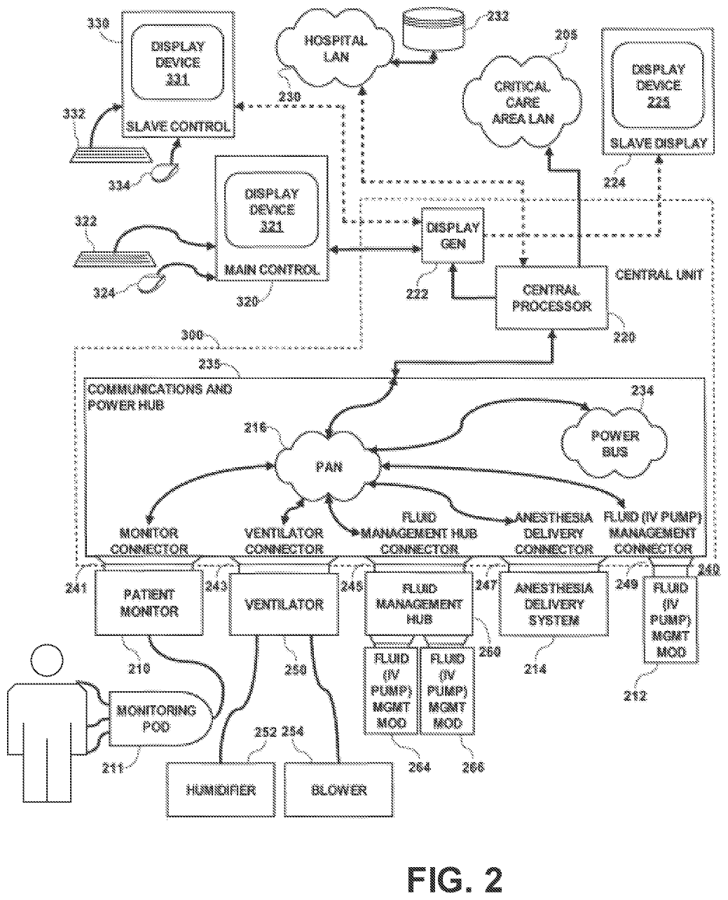

FIGS. 1-2 show an exemplary hospital system. FIG. 1 is a block diagram of a hospital system 200, illustrating four different types of care rooms: an operating room 202, an intensive care unit (ICU) room 204, an emergency room 206, and a critical care room 208. The operating room 202, the ICU room 204 and the emergency room 206 include a patient monitor device as described above. Each patient monitor includes a connection to a critical care area network 205, either directly from the patient monitor or through a base unit (not shown). Each patient monitor also includes patient connections to electrodes or probes attachable to the patient, not shown to simplify the figure. The patient monitors also receive data from other devices and forward that data to critical care area network 205. In the operating room 202, an anesthesia device and fluid management device are coupled to the critical care area network 205 through the patient monitor; in the ICU room a ventilator device and fluid management device are coupled to the critical care area network 205 through the patient monitor; and in the emergency room 206 a ventilator device is coupled to the critical care area network 205 through the patient monitor. In the other critical care room 208 a ventilator device is coupled directly to the critical care area network 205, either directly or through its own base unit.

The operating room 202 includes a patient monitoring module 210 for acquiring and processing signals derived from physiological sensors (not shown) suitable for attachment to a patient. The operating room 202 also includes patient treatment modules: a fluid infusion (IV pump) control and management module 212 and an anesthesia module 214. These modules (210, 212 and 214) are coupled to a central processor 220 via a patient area network (PAN) 216. The central processor 220 is coupled to a display generator 222 which is coupled to a display device 223. The display generator 222 is also optionally coupled to a slave display device 224, as illustrated in phantom. The ICU room 204 includes a monitor module, a fluid management patient treatment module and a ventilator module, coupled to a central processor via a PAN. The emergency room 206 includes a monitor module and a ventilator patient treatment module coupled to a central processor via a PAN. The other critical care room 208 includes a ventilator patient treatment module 208A coupled to the central computer via a PAN 216.

In operation, the PAN 216 may be implemented in any manner allowing a plurality of modules to intercommunicate. For example, the PAN 216 may be implemented as an Ethernet network, either wired or wireless (WLAN). If implemented as a wireless network, it may be implemented according to available standards, such as: (a) a WLAN 802.11b compatible standard, (b) 802.11a compatible standard, (c) 802.11g compatible standard, (d) Bluetooth 802.15 compatible standard, and/or (e) GSM/GPRS compatible standard communication network.

The patient monitoring module 210 corresponds to the operational portion of the patient monitor described above. It receives signals from the physiological sensors attached to the patient, performs the signal processing required to calculate the physiological parameters, and provides that information to the central processor 220 via the PAN 216. Similarly, the patient treatment modules, i.e. the fluid management module 212 and the anesthesia module 214, correspond to the operational portion of the prior art treatment modules described above. The patient treatment modules 212, 214 receive operational data from the central processor 220 via the PAN 216 and in response perform their treatment functions, e.g. monitoring fluids administered to the patient and supplying an anesthetic agent to the patient, respectively. Concurrently, the patient treatment modules 212, 214 send status data to the central processor 220 via the PAN 216. The central processor 220 processes the signals received from the patient monitoring module 210 and the patient treatment modules 212 and 214.

The central processor 220 interacts with the user to receive patient identifier information and treatment instructions and parameters. The central processor 220 configures the patient treatment modules 212, 214 by sending patient identifier information, the treatment instructions and parameters to the patient treatment modules 212 and 214 via the PAN 216.

The patient monitoring and/or treatment modules 210, 212, 214 may include a processor for receiving the configuration parameters from the central processor 220, for controlling the operation of any one or all of modules 210, 212, 214 and for sending status and patient physiological parameter information to the central processor 220 via the PAN 216. The configuration parameters may include, among other things, patient identifier information, set-up parameters, and/or data representing executable instructions for execution by the processor in any one or all of modules 210, 212, 214 in processing data to be provided to the central processor 220. The modules 210, 212, 214, in turn, use the received configuration parameters, and executable instructions in supporting their operation, e.g. for processing data to be provided to the central processor 220.

As described above, there may be more than one central processor 220 in remote locations in the hospital. If a module 210, 212, 214 is disconnected from one central processor 220, then the patient identifier information, the set-up parameters and/or the executable instructions previously sent to it are used to control the operation of that module 210, 212, 214 while it is disconnected. If the disconnected module 210, 212, 214 is reconnected to a central processor 220, possibly in a different location than the central processor 220 from which it is disconnected, then the reconnected module 210, 212, 214 sends data representing the patient identifier information, the operational characteristics of the module, and any patient physiological parameter data gathered while disconnected to the central processor 220 to which it is connected.

The central processor 220 also receives signals representing physiological parameters from the patient monitoring module 210 and possibly from the patient treatment modules 212, 214. These parameters may be relatively standard physiological parameter, such as EKG, heart rate, SpO₂, etc. The central processor 220 may also initiate generation of a new parameter based on signals derived using the patient monitoring module 210 and/or the patient treatment modules 212, 214. For example, the new parameter may be associated with (a) gas exchange, (b) skin color, (c) haemodynamics, (d) pain and/or (e) electro-physiology.

The central processor 220 conditions the display generator 222 to generate signals representing an image for displaying these physiological parameters in an appropriate manner, e.g. a waveform, a status phrase, and/or a number. The display generator 222 is coupled to the display device 223 which displays this image. The display generator 222 may optionally send appropriate image representative signals to the slave display device 224. The slave display device 224 may have a larger, higher resolution screen, or may simply be a display device at a location remote from the location of the central processor. The image generated by the display device 223, under the control of the central processor 220 and display generator 222, may also integrate the display of patient identification, treatment instructions and parameters and status from the patient treatment modules 212, 214 in an appropriate manner. In this manner, information from users as well as patient monitoring modules 210 and patient treatment modules 212, 214 may be integrated into one or more composite images displayed on display devices 223 and 224, for example. Further, the user may tailor the information displayed to suit his or her preferences.

The central processor 220 may also communicate with the central processors of corresponding processing device and display systems in other locations in the hospital, such as those in the ICU room 204, the emergency room 206 and the other critical care room 208 via the critical care area network 205. The central processor 220 may optionally communicate with a central hospital location via a hospital network 230, illustrated in phantom in FIG. 1. In this manner, patient physiological parameters and treatment instructions, parameters and status may be transmitted to a central location and stored in a central storage device 232, also illustrated in phantom.

FIG. 1 illustrates a patient monitoring module 210, and patient treatment modules for fluid management 212, anesthesia control 214, and ventilation control. However, one skilled in the art will understand that there are other monitoring and treatment devices which may include patient treatment modules for control and communication, such as: (a) an incubator, (b) a defibrillator, (c) a warming module, (d) a diagnostic imaging module, (e) a photo-therapy module, (f) a fluid input support module, (g) a fluid output support module, (h) a heart-lung support module, (i) a blood gas monitor, (j) a controllable implanted therapy module, (k) a controllable surgical table and weighing scale, and so forth. Modules for command and communication related to these and other patient treatment devices may be used as illustrated in FIG. 1.

FIG. 2 is a more detailed block diagram illustrating the system illustrated in FIG. 1. In FIG. 2, those elements which are the same as illustrated in FIG. 1 are designated by the same reference number and are not discussed in detail below. FIG. 2 illustrates the system as it would be implemented in one of the rooms 202, 204, 206 or 208 of FIG. 1. In FIG. 2, the central processor 220 and the display generator 222 are comprised within a central unit 300. The central unit 300 is a housing containing the circuitry and connectors necessary to interconnect the central processor 220 and the display generator 222 with: the patient monitoring and patient treatment modules 210, 212, 214, 250 and 260; the display devices 224, 320 and 330; and the multi-patient critical care area LAN 230.

The central processor 220 is coupled to a communications and power hub 235. The communications and power hub 235 comprises the patient area network (PAN) 216 and also a set 240 of module connectors coupled to the PAN 216: e.g. a patient monitor connector 241, a ventilator connector 243, a fluid management hub connector 245, an anesthesia delivery system connector 247 and a fluid (IV pump) management connector 249. The connectors 240 permit the individual modules 210, 212, 214, 250, 260 to be plugged into and removed from the central unit 300 as required. In one embodiment, a user may activate a single mechanical release mechanism to remove a module 210, 212, 214, 250, 260 from the central unit 300 or reattach a module to the central unit 300. The connectors 240 pass data signals between the modules 210, 212, 214, 250, 260 and the central processor 220 via the PAN 216.

The communications and power hub 235 further comprises a power bus 234 for distributing power to the central unit 300. The power bus 234 is further coupled to the PAN 216 for receiving commands from and returning status to the central processor 220. The power bus 234 is also coupled to the connectors 240 (not shown to simplify the figure) to distribute power to the patient monitoring and/or treatment modules 210, 212, 214, 250, 260. In this manner, the central processor 220 may manage the power-on and power-off status of the patient monitoring and treatment modules 210, 212, 214, 250, 260 in accordance with a set of predetermined rules maintained in the central processor 220.

As described above, at least some of the attached modules 210, 212, 214, 250, 260 include circuitry, e.g. batteries, which permit them to continue to operate when disconnected from the central unit 300. When docked, the central processor 220 conditions these modules 210, 212, 214, 250, 260 to transition from operating on battery power to operating on the power supplied by the power bus 234 and recharge their batteries. The internal power supply circuitry of these modules 210, 212, 214, 250, 260 may also supply power supply status information, e.g. current battery capacity, to the central processor 220 through the connectors 240 and PAN 216. The central processor 220 may condition the display generator 222 to generate signals representing an image showing the battery charging condition of the patient monitoring and treatment modules 210, 212, 214, 250, 260 plugged into the central unit 300. This image may be displayed on the display devices 321, 331 and/or 225 in the main control panel 320, slave control panel 330 and/or remote display device 224, respectively.

As described above, the PAN 216 may be implemented as a wireless network. In such an embodiment, the central processor 220 may include a wireless communication interface to the PAN 216. Such an interface enables bidirectional communication with the patient monitoring and treatment modules 210, 212, 214, 250, 260 when they are disconnected from the central unit 300. This communications link enables the central processor 300 to maintain control of the patient monitoring and treatment modules 210, 212, 214, 250, 260 while they are disconnected from the central unit.

Individual patient monitoring and/or treatment modules 210, 212, 214, 250, 260 are coupled to corresponding ones of the connectors 240. For example, a patient monitor module 210 may be plugged into the monitor connector 241, a ventilator module 250 may be plugged into the ventilator connector 243, and so forth. The central unit 300 may include connectors 241, 243, 245, 247, 249 which are specific to the type of patient monitoring or treatment module, 210, 212, 214, 250, 260, expected to be plugged in. Alternatively, the modules 210, 212, 214, 250, 260 may be fabricated with the same type of connector and the connectors 240 may be the same type of matching connectors. In the former embodiment, a particular type of patient monitoring or treatment module 210, 212, 214, 250, 260 may be plugged into a connector 241, 243, 245, 247, 248 corresponding to that type of module. In the latter embodiment, any patient monitoring or treatment module 210, 212, 214, 250, 260 may be interchangeably plugged into any of the connectors 241, 243, 245, 247, 248.

As described above, the patient monitor module 210, plugged into the monitor connector 241, connects to a plurality of electrodes and sensors which may be placed on a patient. A monitoring pod 211 is used to connect the patient-connected electrodes or probes (not shown) to the patient monitor module 210. Similarly a ventilator module 250 may be plugged into the ventilator connector 243. The ventilator module 250, in turn, is coupled to a blower 254 and a humidifier 252. A fluid management hub 260 is plugged into the fluid management hub connector 245. Two fluid (IV pump) management modules 264 and 266 are plugged into the fluid management hub 267. Each fluid (IV pump) management module, 264, 266, is connected to an IV pump (not shown). An anesthesia delivery module is plugged into an anesthesia delivery connector 247. The anesthesia delivery module 214 is connected to a anesthesia delivery device (not shown). An individual IV pump 212 is coupled to an IV pump connector 249. Similar to the other IV pump modules 264 and 266, the fluid (IV pump) management module 212 is connected to an IV pump (not shown).

The central processor 220 is also coupled to the critical care area LAN 205, which, as illustrated in FIG. 1, is coupled to other central units 300 in processing device and display systems in other rooms. The central processor 220 may also be optionally coupled to a hospital LAN 230. The critical care LAN 205 requires real time bandwidth quality-of-service while the hospital LAN 230 requires standard office bandwidth quality-of-service. As described above, if connected to a hospital LAN 230, the central processor 220 may exchange data with a central storage device 232, or any other desired device (not shown) at a remote location in the hospital. Data may be sent from patient monitoring and/or treatment modules 210, 212, 214, 250, 260 to the central storage device 232 through the connectors 240 to the central processor 220 via the PAN 216 and from there to the central storage device 232 via the hospital LAN 230. In addition, control data may be sent in the other direction from the central location to a patient monitoring or treatment module 210, 212, 214, 250, 260.

It is further possible that a central processor 220 in a central unit 300 in a processing device and display system in one treatment room 202, 204, 206, 208 may communicate with a second central processor 220 in a central unit 300 in a processing device and display system in a different treatment room 202, 204, 206, 208 (FIG. 1) via the critical care area LAN 205 or the hospital LAN 230. In this manner, the central processor 220 in one treatment room may control the operation of the second central processor 220 in the second treatment room; may display patient related data received from the second central unit 300 in the different treatment room; and/or may send (a) a patient identifier identifying a particular patient and/or (b) medical information related to the particular patient to the second central processor 220 in the central unit 300 in the second treatment room 202, 204, 206, 208, which receives this information.

It is also possible for the central processor 220 to receive data from one or more of the patient monitoring and/or treatment modules 210, 212, 214, 250, 260, process that data and send control data to one or more of the patient treatment modules 212, 214, 250, 260 in response to the received data, in a manner to be described in more detail below.

The display generator 222 is coupled to a main control panel 320. The main control panel 320 includes a display device 321, a keyboard 322 and a pointing device in the form of a mouse 324. Other input/output devices (not shown) may be fabricated on the main control panel 320, such as: buttons, switches, dials, or touch screens; lights, LCDs, or LEDs; buzzers, bells or other sound making devices, etc. These input/output devices receive signals from and supply signals to the central processor 220, either through the display generator 222, or through separate signal paths, not shown to simplify the figure. The main control panel 320 may be fabricated as a part of the central unit 300, or may be fabricated as a separate unit. The display generator 222 is optionally coupled to a slave control panel 330, which substantially duplicates the functionality of the main control panel 320, but is located remote from the central unit 300. The display generator 222 is also optionally coupled to a slave display device 224. The slave display device 224 includes a display device 225, but does not include any of the other input/output devices included in the main control panel 320 and slave control panel 330.

In operation, the central unit 300 and main control panel 320 provide control and display functions for the patient monitoring and/or treatment modules 210, 212, 214, 250, 260 which are plugged into the common unit 300. A user may manipulate the input devices coupled to the main control panel 320, or slave control panel 330 if available, e.g. the keyboard 322, mouse 324 or other input devices described above. The resulting signals are received by the central processor 220. In response, the central processor 220 sends control signals via the PAN 216 to the patient monitoring or treatment modules 210, 212, 214, 250, 260 which are currently plugged into the central unit 300.

Concurrently, the central processor 220 receives data signals from the patient monitoring and/or treatment modules 210, 212, 214, 250, 260, as described above, and conditions the display generator 222 to produce a signal representing an image for displaying the data from the patient monitoring and/or treatment modules 210, 212, 214, 250, 260, in an appropriate manner. For example, if a patient monitor 210 having the capability of performing an EKG on a patient is plugged into the central unit 300, EKG lead data from the patient monitor 210 is supplied to the central processor 220 through the monitor connector 241 via the PAN 216. The central processor 220, in turn, conditions the display generator 222 to produce signals representing an image of the EKG lead signal waveforms. These image representative signals are supplied to the display device 321 in the main control panel 320, which displays the image of the waveforms of the EKG lead signals. An image representing the heart rate of the patient, derived from the EKG lead signals, may also be similarly displayed in numeric form. Images representing other physiological parameters measured by the patient monitor 210, e.g. blood pressure, temperature, SpO₂, etc. may also be displayed, in an appropriate form, on the display device 321 of the main control panel 320 in a similar manner. The image data may also be displayed on the display device 331 of the slave control panel 330 and on the display device 225 of the slave display 224, if they are available.

In a similar manner, images representing data received from the patient treatment modules, 212, 214, 250, 260, may be displayed on the display devices 321, 331, 225 in an appropriate form. Such data may represent, for example, present settings for the respective treatment modules, such as specified drip rates for IV pumps attached to fluid management modules 212, 264, 266. This data may be represented by images of appropriate form. Such data may also represent physiological parameters which may be measured by the patient treatment devices 212, 214, 250, 260. For example, respiration loops may be displayed in graphical form based on data received from the ventilator module 250, or drip rates for attached IV pumps may be displayed in numerical form based on data received from the fluid management hub 260.

A user may select which physiological parameters to display on the display device 321 and may arrange the location on the display device 321 of the images displaying the selected physiological parameters. In addition, the user may select different physiological parameters to display on the display device 321 in the main control panel 320 than on the display device 331 in the slave control panel 330 and/or on the display device 225 in the slave display 224. Further, the slave display device 224 may have a display device 225 which is larger and/or higher resolution than those in the main control panel 320 and the slave control panel 330, so that the images may be more easily seen, and/or may be displayed at an increased resolution.

The central processor 220 may also receive data from the power bus 234 via the PAN 216 representing the state of the power supplies in the patient monitoring and treatment modules 210, 212, 214, 250, 260. The central processor 220 may, for example, condition the display generator 222 to generate a signal representing an image representing the current charge condition of the respective batteries in the patient monitoring and treatment modules 210, 212, 214, 250, 260 plugged into the central unit 300, either separately or in composite, based on data received from the power bus 234. Further, the patient monitoring and/or treatment modules 210, 212, 214, 250, 260 may provide data to the central processor 220 indicating an error condition in the module. The central processor 220 may condition the display generator 222 to generate a signal representing an image showing the user the error condition of that module.

The central processor 220 may also produce signals for controlling the operation of the other output devices on the main and slave control panel 320, 330, described above. For example, the central processor 220 may analyze the physiological parameters derived from signals received from the patient monitoring and/or treatment modules 210, 212, 214, 250, 260 to determine if any limits have been exceeded. This may entail separately calculating and verifying each physiological parameter response determined from a patient monitoring and/or treatment module, and comparing it to a predetermined parameter range to determine if it exceeds a limit, or analyzing more than one physiological parameter to determine if a function of those physiological parameters exceeds a limit. If a limit has been exceeded, then the central processor 220 may condition the output devices on the main and slave control panel 320, 330 to provide an alarm. For example, the central processor 220 may generate a signal which activates a light, a buzzer, a bell and/or other such device on the main control panel 320, and/or the slave control panel 330, if available, to produce a visible or an audible alarm. The central computer 220 may also send a signal over the critical care area LAN 205 and/or the hospital LAN 230 indicating that a limit has been exceeded. A similar alarm may be generated at the remote location in response to the receipt of this signal.

In another embodiment, the patient monitor 210 may analyze the physiological parameters derived from signals received from the patient monitoring and/or treatment modules 210, 212, 214, 250, 260 to determine if any limits have been exceeded and provide an alarm or other notification. The alarm may generate an alarm signal that is sent to a bridge or nursing station (such as slave control 330 of FIG. 2). Similarly, a use may be able to configure parameter settings at the bridge or nursing station and generate a parameter settings signal that is transmitted to the patient monitor module 210. In yet another embodiment, another device on the system, such as the ventilator 250 or the anesthesia delivery system 214 may process the physiological parameters and send the alarm signal to the bridge or nursing station.

In one exemplary embodiment, the hospital system 200 is programmed to monitor a predetermined set of patient parameters that are indicative of the presence of a particular medical condition, store predetermined criteria for each parameter, and provide a notification that is indicative of a preliminary automated diagnosis of the medical condition if all of the criteria are met.

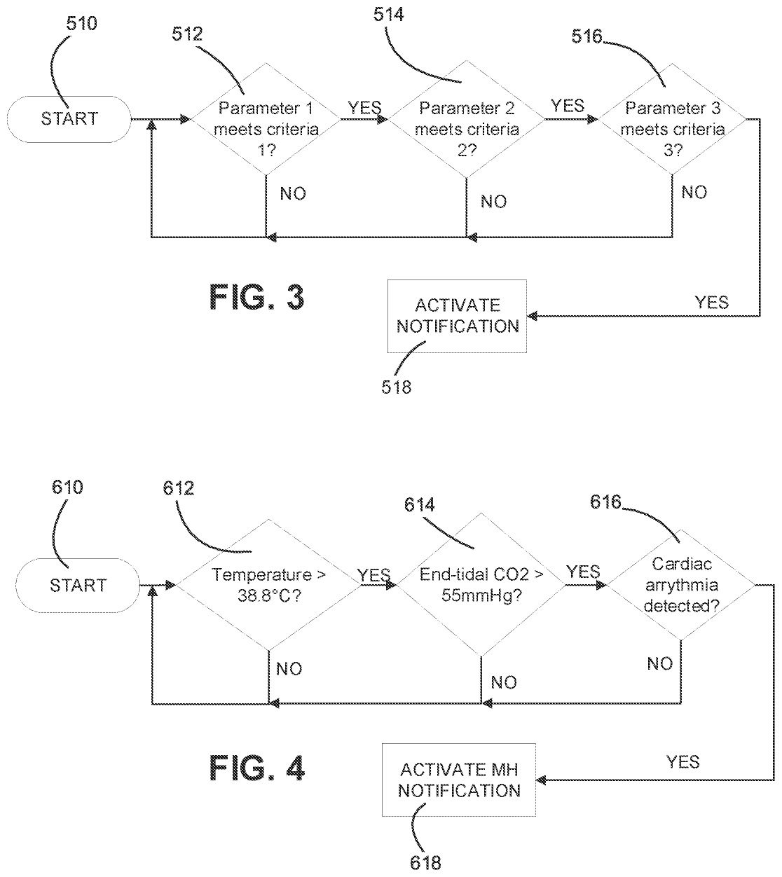

The method by which such a notification could be displayed is represented visually in FIG. 3. Monitoring of the set of predetermined parameters begins at Step 510, which can be initiated either manually (by user input) or automatically by the start of another process in the hospital system 200. In this case, three patient physiological parameters (Parameters 1, 2 and 3) are being continuously monitored or monitored with pre-determined intervals, depending on the types of the physiological parameters, the medical condition of the patient and/or where the patient is located. For purposes of this method, “monitored for pre-determined intervals” means that a sensor signal is received at a clinically acceptable frequency for that patient physiological parameter. For example, fora patient located in an operation room where the patient is connected to multiple physiological sensors and a patient monitor, SpO₂, electrocardiogram (ECG) and temperature may be continuously monitored, and non-invasive blood pressure (NIBP) may be monitored with pre-determined intervals (e.g., every 3-5 minutes). A notification is activated (Step 518) only if all three of Parameters 1, 2 and 3 meet a predetermined criteria assigned to each parameter (Steps 512, 514, 516). Accordingly, if Parameter 1 meets Criteria 1 but Parameter 2 does not meet Criteria 2, no notification is activated. It should be noted that, in other embodiments, any number of parameters could be monitored.

The set of predetermined parameters that are monitored in the method shown in FIG. 3 are each preferably selected from the group of patient physiological parameters (such as SpO₂ or internal temperature), operating parameters of the hospital system 200 (such as activation of gas flow in the anesthesia module 214), laboratory test results, and patient medical history. Laboratory test results and patient medical history information would likely need to be entered manually by hospital staff.

An example of the application of this method to provide a preliminary automated diagnosis for malignant hyperthermia (“MH”) is provided in FIG. 4. MH is a hypermetabolic response in humans to potent inhalation agents (such as halothane, sevoflurane, desflurane), the depolarizing muscle relaxant succinylcholine, and rarely, to stresses such as vigorous exercise and heat. Most patients who are to MH susceptible have no phenotypic changes without anesthesia. Accordingly, it is impossible to diagnose susceptibility without either the exposure to the “trigger” anesthetics or by specific diagnostic testing.

The following are generally accepted criteria for diagnosing MH:

Respiratory acidosis—End-tidal CO₂>55 mmHg; PaCO₂>60 mm Hg;

Cardiac involvement—Unexplained sinus tachycardia, ventricular tachycardia or ventricular fibrillation;

Metabolic acidosis—Base deficit >8 m/Eql, pH<7.25;

Muscle rigidity—Generalized rigidity; severe masseter muscle rigidity;

Muscle breakdown—Serum creatine kinase concentration >20,000/L units; cola colored urine; excess myoglobin in urine or serum; plasma [K+]>6 mEq/L;

Temperature increase—Rapidly increasing temperature; T>38.8 degrees C.;

Other—Rapid reversal of MH signs with dantrolene. Elevated resting serum creatine kinase concentration; and

Family history—Central Core Disease (CCD) and Multi-Minicore Disease (MmCD) predisposes patients to episodes of MH.

Existing patient monitoring systems can continuously measure end-tidal CO₂, cardiac involvement (via arrhythmias and heart rate), and internal patient temperature, and can detect or infer the presence of inhalation anesthetic agents. The combination of these parameters can provide an early preliminary diagnosis of MH and, if brought to the attention of hospital staff, enable staff to confirm (or disprove) the preliminary diagnosis by checking for the presence of criteria that cannot be monitored by the hospital system 200.

Referring again to FIG. 4, monitoring for MH could begin (Step 610) when a patient is exposed to an inhalation anesthetic agent. If the patient is in the operating room 202 of the hospital system 200, this could occur when the anesthesia module 214 begins the flow of an inhalation anesthetic agent to the patient (See FIG. 1). Alternatively, monitoring could start (Step 610) when the presence of the inhalation anesthetic agent is detected by a sensor connected to the patient monitoring module 210. As used in this application, the term “inhalation anesthetic agent” is intended to refer to an agent that is inhaled by the patent (most commonly using in a vaporized form) and provided in a sufficient dosage to render a patient unconscious. Common inhalation anesthetic agents include halothane, isoflurane, desflurane, sevoflurane. These inhalation anesthetic agents are often delivered as a vapor using a carrier gas such as a mixture of nitrous oxide and oxygen or a mixture of oxygen and nitrogen.

The hospital system 200 then continuously monitors internal patient temperature (Step 612), end-tidal CO₂ (Step 614), and cardiac function (Step 616). For internal patient temperature, the criteria is considered having been met (represented as a “YES” outcome of Step 612) if the patient's internal temperature is greater than 38.8 degrees C. Optionally, this criteria may also require a rapid increase in temperature, e.g., an increase in temperature of at least 1 degree C. within a one hour period. For end-tidal CO₂, the criteria is considered having been met (represented as a “YES” outcome of Step 614) if the patient's end tidal CO₂ is measured at greater than 55 mmHg. For cardiac function, the criteria is considered having been met (represented as a “YES” outcome of Step 616) if an arrhythmia (sinus tachycardia, ventricular tachycardia or ventricular fibrillation) is detected by the patient monitor. If all three criteria of Steps 612, 614, 616 are simultaneously met, then an MH notification is activated (Step 618). Continuous monitoring could continue until the notification is activated, fora predetermined period of time (e.g., 24 hours), or until manually terminated by hospital staff.

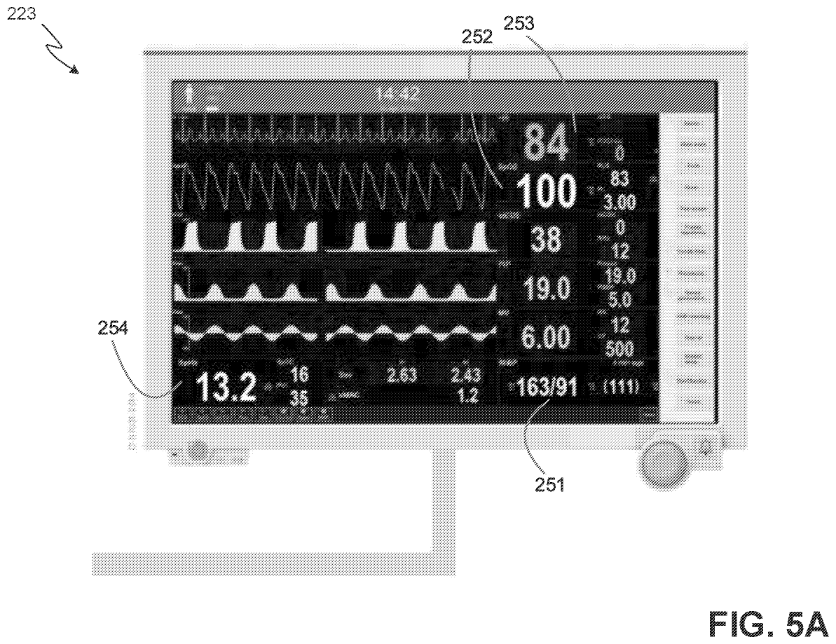

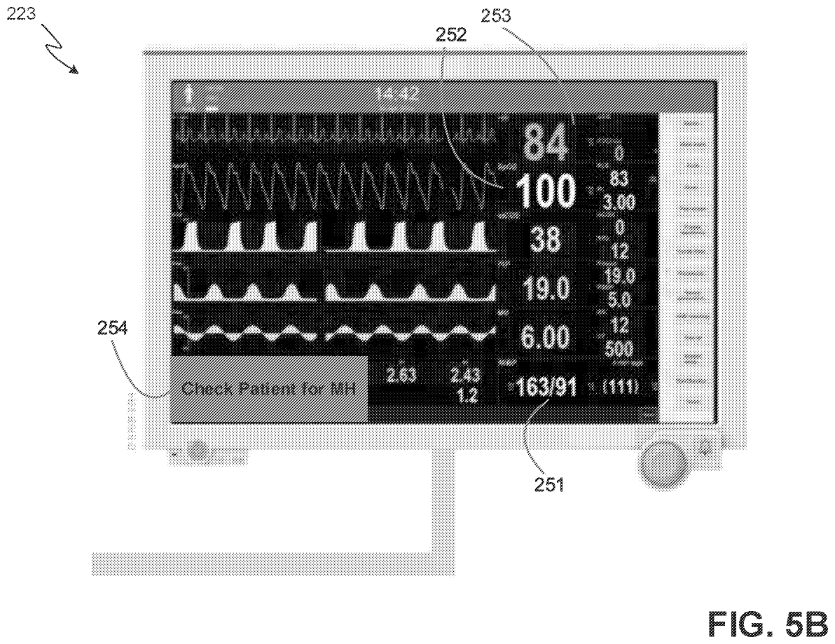

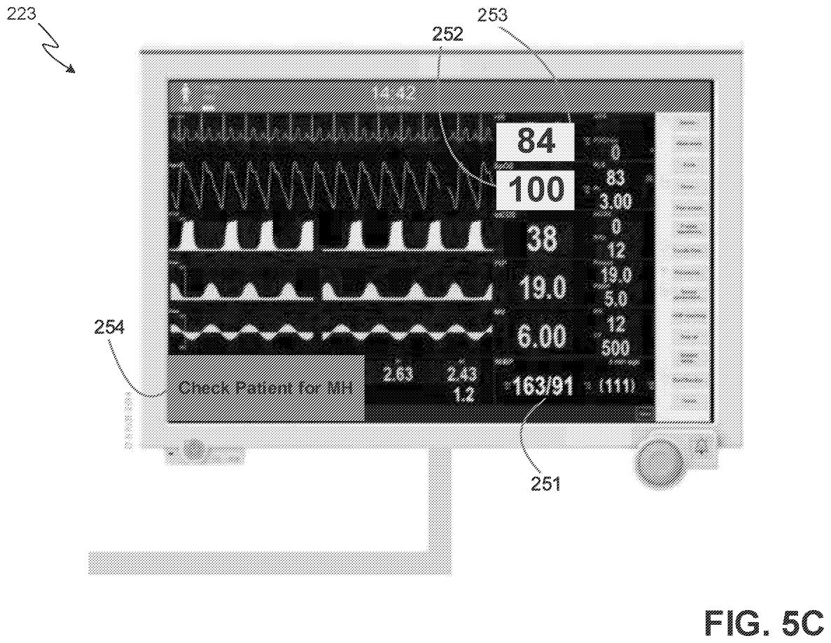

FIGS. 5A-5C show an example of how a notification indicating a preliminary automated MH diagnosis could be provided on the display device 223. In FIG. 5A, the display device 223 shows a plurality of patient physiological parameters, each in a window. For example, blood pressure is displayed in window 251 and SpO₂ is displayed in window 252. In this embodiment, a window 254 allows for cardiac output, wedge pressure and other user interactions to occur. When the conditions for MH are met, the notification is provided by changing the background color of window 254 to red, as shown in FIG. 5B. Any other suitable method of providing a notification, as discussed above, could be used.

Optionally, when all criteria are met, the patient parameters shown on the display device 223 the windows displaying parameters that are used to determine the preliminary automated MH diagnosis can also be visually emphasized, so that hospital staffs' attention is drawn to those parameters. In this case, the visual emphasis is provided by changing the backgrounds of windows 252 (SpO2) and 253 (heart rate/EKG) to yellow. In another non-limiting example, the visual emphasis is provided by highlighting at least one of the physiological parameters monitored by the patient monitor and other medical information displayed on the device 223 associated with the patient, for example, laboratory results and medical history stored in an electronic medical record (EMR) system.

All examples and conditional language recited herein are intended for pedagogical purposes to aid the reader in understanding the principles of the present invention and the concepts contributed by the inventor in furthering the art. As such, they are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It is to be understood that the embodiments described herein are merely exemplary and that a person skilled in the art may make many variations and modifications without departing from the spirit and scope of the invention. All such variations and modifications are intended to be included within the scope of the invention, as defined by the following claims.

1. A method for identifying a medical condition of a patient, the method comprising:
(a) monitoring a predetermined set of physiological parameters of the patient;
(b) receiving at least one additional parameter of the patient that cannot be measured by a sensor attached to a patient care system; and
(c) providing a notification on the patient care system when each of the parameters of the predetermined set of physiological parameters and the at least one additional parameter meets a predetermined criteria;
wherein the notification identifies the medical condition, and the predetermined set of physiological parameters and the at least one additional parameter each meeting the predetermined criteria is indicative of the presence of the medical condition in the patient.

2. The method of claim 1, wherein step (a) comprises monitoring a predetermined set of parameters of the patient consisting of a plurality of parameters selected from the group consisting of SPO₂, EKG, heart rate, electro-physiology, gas exchange, skin color, and hemodynamics.

3. The method of claim 1, wherein the medical condition includes at least one selected from the group of malignant hyperthermia, pulmonary embolism, and shock.

4. The method of claim 1, wherein the at least one additional parameter of step (b) includes at least one selected from the group consisting of family medical history, response to administration of a medication, and results of a laboratory test.

5. The method of claim 4, wherein the identification of malignant hyperthermia is in response to detection of at least two selected from the group of: an end-tidal Co2 of greater than 55 mmHg, an internal temperature greater than 38.8 degrees C., and a cardiac arrhythmia.

6. The method of claim 1, further comprising, initiating steps (a) and (b) when the patient is exposed to an inhalation anesthetic agent.

7. The method of claim 1, further comprising, initiating step (a) and (b) when an anesthesia module begins to supply an inhalation anesthetic agent to the patient.

8. The method of claim 7, further comprising, continuing to perform step (a) for a predetermined period of time after the anesthesia module begins to supply an inhalation anesthetic agent to the patient.

9. The method of claim 1, wherein step (c) further comprises providing a notification that directs a user to check the patient for at least one additional parameter that is indicative of the presence of the medical condition.

10. (canceled)

11. The method of claim 1, further comprising:
(d) simultaneously with the performance of step (c), visually emphasizing on a display of the patient monitoring system representations of each of the parameters of the predetermined set of parameters that meets the predetermined criteria.

12. A method comprising:
(a) monitoring a predetermined set of parameters comprising (i) end-tidal CO2 of a patient, (ii) an internal temperature of the patient, (iii) a cardiac rhythm of the patient, and (iv) data indicative of an inhalation anesthetic agent being administered to the patient; and
(b) activating a notification on a patient care system when each of the parameters of the predetermined set of parameters meets a predetermined criteria, the notification providing an indication that the patient may be experiencing malignant hyperthermia.

13. The method of claim 12, wherein monitoring an internal temperature of the patient comprises monitoring for an internal temperature that exceeds 38.8 degrees C. or monitoring for an increase of at least 1.0 degrees C. in internal temperature within one hour.

14. The method of claim 12, wherein step (b) comprises activating the notification on the patient care system when each of the parameters of the predetermined set of parameters meets the predetermined criteria, the notification advising a user to check for at least one additional parameter that indicates that the patient is experiencing malignant hyperthermia, the at least one additional parameter comprises a parameter that cannot be measured by a sensor attached to the patient care system.

15. The method of claim 12, wherein the predetermined criteria for each parameter is as follows:
(i) end-tidal CO₂ of the patient is greater than 55 mmHg;
(ii) internal temperature of the patient is greater than 38.8 degrees C. or internal temperature increase of at least 1.0 degrees C. within an hour;
(iii) an arrhythmia is detected in the cardiac rhythm of the patient; and
(iv) data indicating that an inhalation anesthetic agent has been administered to the patient within the last 24 hours.

16. A patient monitoring system comprising:
a patient monitor module having at least one sensor for monitoring a physiological parameter of a patient;
a display; and
at least one processor that is electrically connected to the patient monitor and the display, the at least one processor being adapted to (a) monitor a predetermined set of physiological parameters measured by the at least one sensor and at least one additional parameter that is not capable of being measured by the at least one sensor, and (b) provide a notification through the display when each of the parameters of the predetermined set of parameters meets a predetermined criteria;
wherein the notification identifies a medical condition, and the predetermined set of parameters each meeting the predetermined criteria is indicative of the presence of the medical condition in the patient.

17. The patient monitoring system of claim 16, wherein the predetermined set of parameters consists of a plurality of parameters selected from the group consisting of physiological parameters of the patient, laboratory test data from the patient, operating parameters of a hospital system, and patient medical history.

18. The patient monitoring system of claim 16, wherein the medical condition includes at least one selected from the group of malignant hyperthermia, pulmonary embolism, and shock.

19. The patient monitoring system of claim 18, wherein the medical condition is malignant hyperthermia.

20. The patient monitoring system of claim 19, wherein the predetermined criteria consists of at least two selected from the group of: an end-tidal Co2 of greater than 55 mmHg, an internal temperature greater than 38.8 degrees C., and a cardiac arrhythmia.

21. The patient monitoring system of claim 16, further comprising an anesthesia module and wherein the at least one processor is adapted to initiate monitoring of the predetermined set of physiological parameters when the anesthesia module commences flow of an inhalation anesthetic agent to the patient.

22. The patient monitoring system of claim 21, wherein the at least one processor is adapted to continue monitoring of the predetermined set of physiological parameters for a predetermined period of time after the anesthesia module commences flow of an inhalation anesthetic agent to the patient.

23. The patient monitoring system of claim 16, wherein the notification directs a user to check the patient at least one additional parameter that is indicative of the presence of the medical condition.

24. (canceled)

25. The patient monitoring system of claim 16, wherein the at least one processor is adapted to cause representations of each of the physiological parameters of the predetermined set of parameters that meets the predetermined criteria to be visually emphasized on the display when the notification is being displayed.

26. The method of claim 9, wherein the at least one additional parameter comprises at least one selected from the group of: laboratory test data from the patient, operating parameters of a hospital system, and patient medical history.

27. The method of claim 12, wherein step (a) comprises monitoring a predetermined set of parameters consisting of (i) end-tidal CO2 of a patient, (ii) an internal temperature of the patient, (iii) a cardiac rhythm of the patient, and (iv) data indicative of an inhalation anesthetic agent being administered to the patient.
